(19) United States
(12) Patent Application Publication
(10) Pub. No.: US 2017/0150984 A1
(43) Pub. Date: Jun. 1, 2017

(54) CUTTING DEVICE WITH PRECISE CUTTING DEPTH

(71) Applicant: Cook Medical Technologies LLC, Bloomington, IN (US)

(72) Inventor: Liam Breen, Limerick (IE)

(73) Assignee: Cook Medical Technologies LLC, Bloomington, IN (US)

(21) Appl. No.: 15/363,301

(22) Filed: Nov. 29, 2016

Related U.S. Application Data
(60) Provisional application No. 62/261,531, filed on Dec. 1, 2015.

(51) Int. Cl. A61B 17/3211

(52) U.S. Cl. CPC A61B 17/3211; A61B 2017/320052

(57) ABSTRACT

An apparatus for at least partially cutting a tissue layer, the apparatus including an elongated body with a distal portion, a tissue-engaging surface at the distal portion, and a knife located proximate to the tissue-engaging surface. The knife may have an active state, where in the active state, the knife has a cutting portion extending a first distance away from the tissue-engaging surface, and where the first distance corresponds to a depth of a desired cut within the tissue layer.

### CROSS-REFERENCE TO RELATED APPLICATIONS

This application claims priority to U.S. provisional patent application Ser. No. 62/261,531, filed Dec. 1, 2015, which is incorporated by reference herein in its entirety.

### BACKGROUND

There are several disorders of the gastrointestinal tract, e.g., gastrointestinal inflammation, gastrointestinal cancer, gastrointestinal infection, gastrointestinal motility dysfunction, or lesions, wounds or contusions of tissue of a portion of the gastrointestinal tract that can cause gastrointestinal lesions. In addition, there is a wide variety of medical procedures that require removal or dissection of the mucosal or submucosal layers of gastrointestinal tract wall to treat these disorders.

To treat motility disorders from within the esophagus, a procedure called per-oral endoscopic myotomy (“POEM”) may be used. In this procedure, a tunnel is generally formed beneath the mucosal and submucosal layers such that a delivery device (e.g., a gastroscope with a working channel) may access the underlying layers of muscle tissue. The top muscle layer may then be cut in the axial direction, which may weaken tightness in the esophagus to treat the motility disorder.

Currently, a high level of skill and attention is required to make the cut in the upper muscle layer in isolation, ensuring the lower muscle layer remains intact. The target area where it is desirable to perform the cut is near vital arteries and is generally at a location in a position of the body that is difficult to reach, and therefore it is difficult for a medical professional to control the depth and direction of the cut. Accordingly, it would be desirable to provide an improved cutting device configured to perform a cut with a precisely-controlled position and depth in a muscle layer of tissue within a human or animal body.

### DESCRIPTION

An apparatus for at least partially cutting a tissue layer, the apparatus comprising an elongated body with a distal portion, a tissue-engaging surface at the distal portion and a knife located proximate to the tissue-engaging surface and having an active state, wherein in the active state, the knife has a cutting portion extending a first distance away from the tissue-engaging surface, and wherein the first distance corresponds to a depth of a desired cut within the tissue layer.

The knife may comprise a deflectable portion located proximally of its distal end, and wherein the deflectable section comprises at least a portion of the cutting section.

The tissue-engaging surface may face substantially perpendicular to a longitudinal direction of the distal portion of the elongated body.

The knife may be adjustable to a second state, wherein in the second state, the cutting portion extends a second distance from the tissue-engaging surface corresponding to a second depth of a desired cut within the tissue layer.

The knife may comprise an inactive state wherein the knife does not extend from the tissue-engaging surface.

A stability device may extendable away from the body and be configured to orient the tissue-engaging surface towards the tissue layer.

The stability device may comprise at least one wing configured to extend away from the elongated body.

The apparatus may have at least one expander located at the distal portion opposite of the tissue-engaging surface, the expander configured to provide a force for pushing the tissue-engaging surface towards the target tissue.

A lumen may extend to the distal portion, the lumen being configured to inject a fluid.

### BRIEF DESCRIPTION OF THE DRAWINGS

FIG. 1A is a side view of an embodiment of a medical device comprising a cutting device engaged with a target location in a body in accordance with the present invention.

FIG. 1B is a front view of an embodiment of a medical device comprising a cutting device engaged with a target location in a body.

FIG. 2A is a side view of an embodiment of a cutting device having a knife in an active state in accordance with the present invention.

FIG. 2B is a side view of an embodiment of a cutting device having a knife in an inactive state in accordance with the present invention.

FIG. 2C is a front view of an embodiment of a cutting device having an adjustable knife extending a first distance in accordance with the present invention.

FIG. 2D is a front view of an embodiment of a cutting device having an adjustable knife extending a second distance in accordance with the present invention.

FIG. 3A is a perspective view of an embodiment of a cutting device having stability wings in an expanded state in accordance with the present invention.

FIG. 3B is a perspective view of an embodiment of a cutting device having stability wings in a retracted state in accordance with the present invention.

FIG. 4A is a side, partially cutout view of an embodiment of a medical device with a cutting device in operation within a submucosa tunnel within the wall of the esophagus of a patient, wherein the cutting device has an expander providing a downward force on the cutting device in accordance with the present invention.

FIG. 4B shows a cutout view of an esophagus of a patient prior to formation of a submucosal tunnel.

### DETAILED DESCRIPTION

The embodiments herein are described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

The invention is defined by the claims, may be embodied in many different forms, and should not be construed as limited to the embodiments set forth herein; rather, these embodiments are provided so that this disclosure will be thorough and complete, and will fully convey enabling disclosure to those skilled in the art. As used in this specification and the claims, the singular forms “a,” “an,” and “the” include plural referents unless the context clearly dictates otherwise. The terms “proximal” and “distal” are used herein in the common usage sense where they refer respectively to a handle/doctor-end of a device or related object and a tool/patient-end of a device or related object. The term “about” when used with reference to any volume, dimension, proportion, or other quantitative value is intended to communicate a definite and identifiable value within the standard parameters that would be understood by one of skill in the art (equivalent to a medical device engineer with experience in the field of tissue devices and/or pressure/vacuum-exertion and monitoring devices), and should be interpreted to include at least any legal equivalents, minor but functionally-insignificant variants, and including at least mathematically significant figures.

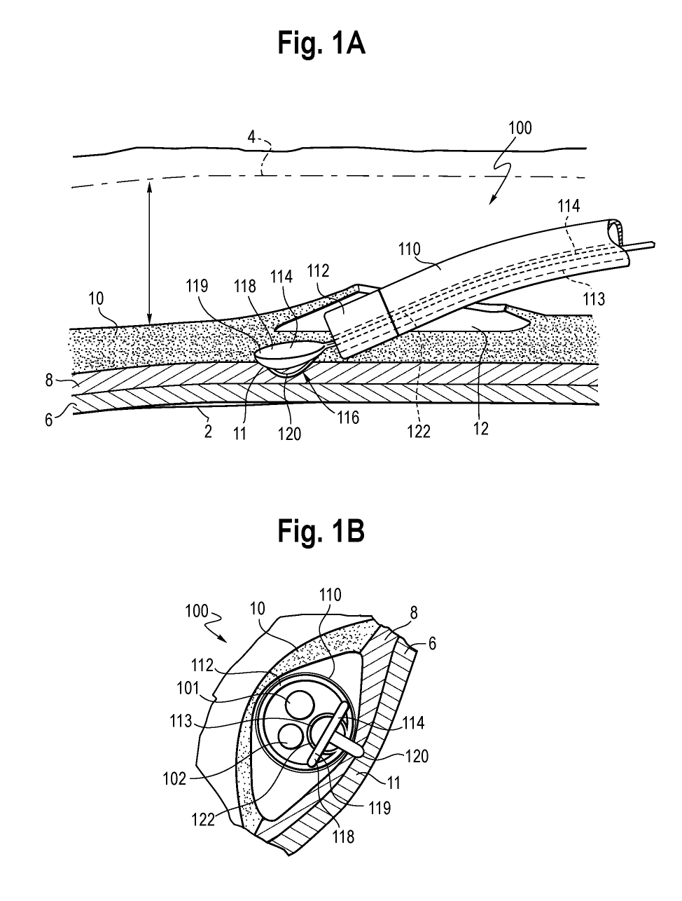

FIG. 1A depicts one embodiment of a medical device 100 configured to at least partially cut a tissue layer, such as a top muscle layer 8 underlying the mucosal and submucosal layers 10 inside an esophagus. Referring to FIG. 1A, the medical device 100 with a delivery device 110 is within an esophagus 2 (with centerline 4) of a patient. The delivery device 110 may be, for example, an endoscope or gastroscope and may comprise a cap 112 on its distal end. The cap 112, as shown, is configured to house at least a portion of a cutting device 114. In some embodiments, a cap is not necessary. As depicted, at least a proximal portion of the cutting device 114 may extend through a working channel 113 of the delivery device 110 such that it can be controlled from a proximal location (not shown) of the delivery device 110.

Referring to FIG. 1B, the delivery device 110 may house other devices configured to perform or facilitate a medical procedure, such as devices 101 and 102, which may correspond to a light and a camera, along with the cutting device 114. Devices 101 and 102 may be located at least partially within the working channel 113, may be integral with the delivery device 110, and/or may otherwise be attached to the delivery device 110.

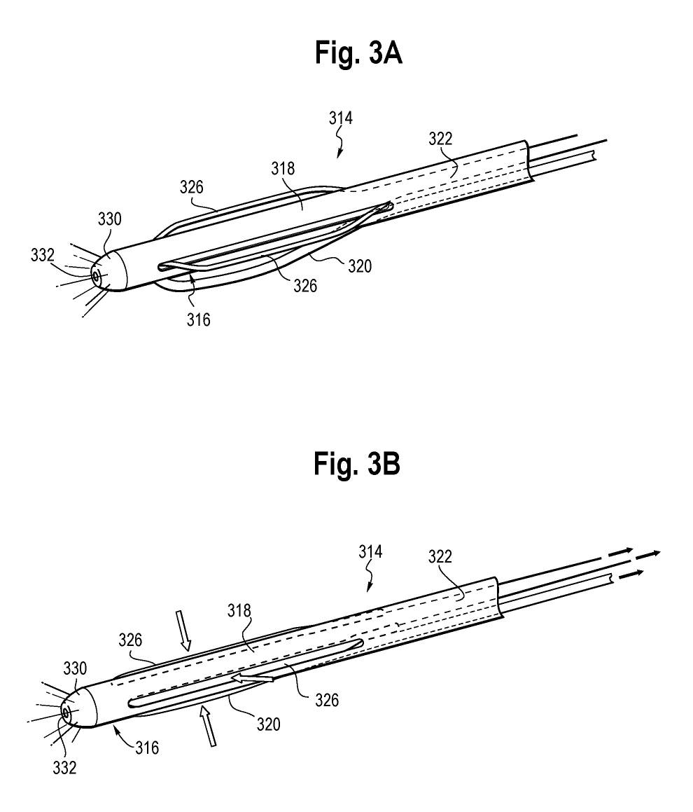

As best shown by FIG. 1A, the cap 112 may be in situ with respect to a target location within the esophagus 2 when located within a submucosal tunnel 12, which may extend within or under the mucosal and submucosal layers 10. Underlying muscle layers 8 (the “top” muscle layer) and 6 (the “bottom” muscle layer) may underlie the mucosal and submucosal layer 10. For purposes of illustration, FIG. 5 depicts the esophagus 2 prior to the deployment of the medical device 100, the esophagus 2 having the top muscle layer 8, the bottom muscle layer 6, and the submucosal layers 10 (which may be under a mucosal layer, not shown). As depicted, a target tissue area 11 may be the target area for an incision to be made in a muscle layer. Referring to FIG. 1A, the top muscle layer 8 may comprise the target tissue area 11 for desired cutting by the cutting device 114. The cutting device 114 may comprise an elongated body 122 at least partially within the working channel 113, and when in operation, a distal portion 118 of the cutting device 114 may extend distally beyond the terminal end of the working channel 113 such that the distal portion 118 engages the target tissue area 11. The distal portion 118 of the cutting device 114 may comprise a tissue-engaging surface 116 and a knife 120. The knife 120, as depicted, may be located proximate or adjacent to, and extend from, the tissue-engaging surface 116. The knife 120 may include, for example, a cauterizing knife (e.g., a tool such as a deflectable wire, wherein the wire may be configured to heat up when electrically charged to thereby cauterize nearby tissue), a knife with a sharp surface, a wire saw or other moveable cutter, or any other suitable cutting device. In the depicted embodiment, the knife 120 and the tissue-engaging surface 116 are located on a stabilizing platform 119 at the distal portion 118 of the cutting device 114, though this is not necessary in all embodiments (for example, see the embodiment of FIG. 3A).

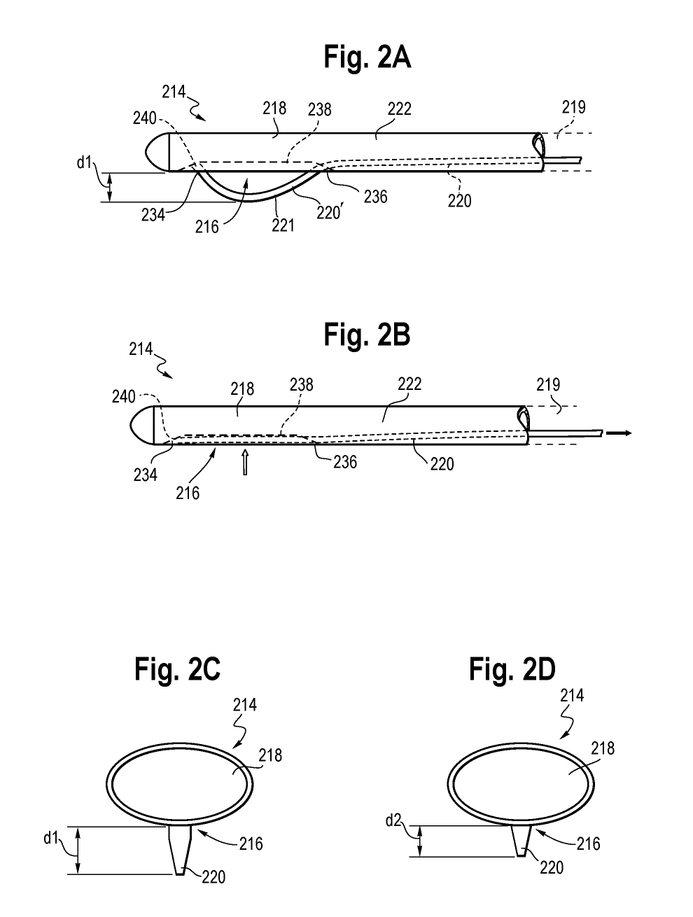

Referring to FIGS. 2A-2B, an embodiment of a cutting device 214 is shown. The cutting device 214 may be delivered to the treatment site using an endoscope such as a gastroscope described above with reference to FIG. 1A. The cutting device 214 includes a knife 220 on an elongated body 222. The knife 220 may be deflectable. The knife 220 may have an active state (e.g., a cutting state of FIG. 2A) and an inactive state (e.g., a non-cutting state of FIG. 2B). For example, referring to FIG. 2A, the cutting device 214 is depicted with the knife 220 being deflected such that it extends a first distance d1 from a tissue-engaging surface 216 of the cutting device 214. The first distance d1 may correspond with (but not necessarily be equal to) a depth of a desired cut within a tissue layer (see FIG. 2C). The tissue-engaging surface 216 may be configured to contact the target tissue layer at the top of the layer 8 (see FIG. 1A) such that the knife 220 extends a distance dl into the layer, (as best shown by tissue-engaging surface 116 in FIG. 1A-1B). The tissue-engaging surface 216 may be a smooth surface, may be lubricated, and/or may be configured to easily slide with respect to a tissue layer in at least the direction of a desired cut. In some embodiments, the tissue-engaging surface 216 may not be located on the elongated body 222, but rather may be comprised on a separate component extending from the elongated body 222. Further, the tissue-engaging surface 216 may face substantially perpendicular to the longitudinal direction of the distal portion 218 of the elongated body 222.

FIG. 2B shows the cutting device 214 with the knife 220 in an inactive state. In this state, the knife 220 may be at least partially confined within a distal portion 218 (e.g., in a groove 238) of the cutting device 214 such that the knife 220 does not extend from the distal portion 218. In this state, the knife 220 is substantially unable to perform a cutting operation. This embodiment is advantageous when, for example, it is only desirable for the cutting device 214 to be selectively operational, which may allow a user to safely navigate sensitive areas with the cutting device 214 in an inactive state and then selectively changing to an active state when reaching a target area. It is noted that this advantage may be achieved by other embodiments, such as an embodiment with a cauterizing knife, where selectively providing and cutting off current to a wire defining a cauterizing knife may achieve an active and inactive state. The embodiment of FIGS. 2A-2B may additionally be advantageous, as the cutting device 214 occupies a smaller space when inactive than when active, which may, for example, save space within a delivery device (e.g., a gastroscope) during delivery to the target area.

The deflection of the knife 220 noted above may be achieved in a variety of ways. For example, in one embodiment, the knife 220 may comprise a shape memory metal such as nitinol (NiTi), which may deflect to extend from the distal portion 218 of the cutting device 214 when the knife 220 is positioned at the target area. In other embodiments, the deflection may be achieved by feeding an additional amount of wire (where the wire defines the knife 220) towards the distal portion 218, thereby causing the knife 220 to controllably deflect (e.g., bow or bend), thereby forming a cutting portion 221 extending from the distal portion 218. The deflected portion 220′ of the knife 220 comprises at least a portion of the cutting portion 221, which corresponds with the area of the knife 220 that will penetrate the target tissue during a cut. The distal end 240 of the knife 220 may be anchored within the distal portion 218 of the elongated body 222 (potentially near attachment point 234), as shown in FIG. 2A, and therefore the deflected portion 220′ of the knife 220 is located proximally of the distal end 240. Other suitable methods or devices configured to achieve deflection may be used. The distal portion 218 may, in some embodiments, include the groove 238 configured to house the knife 220 when in an inactive state. Attachment points 234 and 236 may exist at each side of the groove 238, creating entry and exit ports for the knife 220 which may provide support and control of the deflection of the knife 220. In other embodiments, the knife 220 may remain outside the elongated body 222 in all states and/or permanently in a deflected state.

The extent of deflection of the knife 220 may be adjustable. Referring to FIGS. 2C-2D, the knife 220 may be adjustable to achieve different distances d1 and d2 corresponding to different cutting depths. The proper cutting depth may be selected based on the thickness of the target tissue layer, the severity of the medical disorder, the proximity to vital organs or sensitive areas within the body, etc. The cutting depth is preferably controlled by the medical professional from a location outside the body, for example from the proximal end of the medical device. It may be controlled by, for example, controlling the “bow” or “bend” in the wire defining the portion extending from the tissue-engaging surface 216 by selectively precisely controlling the above-described degree of proximal feeding of a wire comprised by the knife 220 using any suitable method. Other methods and devices for achieving this described adjustment may be used. The knife 220 may comprise any number of cutting distances or depths. These embodiments are advantageous, as they provide a medical professional with a precise (and potentially variable/selectable) cutting depth that may reduce the skill and attention required when performing a cutting procedure and reduce the probability of complications or damage to vital and/or fragile areas in the body. This is important where there is a small difference between an insufficiently shallow cut and a cut extending too far into or through the bottom muscle layer 6.

In some embodiments, it may be desirable to provide stabilization or rotation control such that the knife does not damage the fragile submucosal layer and/or other fragile nearby areas. Referring to FIG. 3A, an embodiment of a cutting device 314 comprises a knife 320 deflected from a distal portion 318 of an elongated body 322, and particularly deflected radially away from a tissue-engaging surface 316 with respect to a longitudinal axis of the elongated body 322. In the embodiment shown in FIGS. 3A and 3B, the cutting device 314 further comprises stability wings 326, which may be configured to prevent rotation of the cutting device 314 and/or orient the tissue-engaging surface 316 towards the target area. The stability wings 326 extend radially outward from the elongated body 322. The stability wings 326 may extend, for example, in a direction approximately perpendicular from the direction that the knife 320 extends away from the elongated body 322. In operation, the stability wings 326 may work by, for example, filling space between two layers of tissue (e.g., between the top muscle layer 8 and the mucosa and submucosa layers 10 in FIG. 1A). In other words, the stability wings 326 may extend outward from the elongated body 322 and engage one or more layers of tissue to prevent problematic rotation of the cutting device 314 such that the knife 320 and/or tissue-engaging surface 316 remains in contact with a target area. The stability wings 326 may, particularly when acting between two layers of tissue, provide a downward force such that the tissue-engaging surface 316 and/or the knife 320 are in sufficient engagement with the target area.

The described stability wings 326 may have expanded and retracted states. FIG. 3A shows the stability wings 326 in an expanded state, where the stability wings 326 extend a distance away from the elongated body 322. The stability wings 326 may further comprise a retracted state as shown in FIG. 3B, wherein the stability wings 326 are retracted (and potentially retracted to a position at least partially within the elongated body 322). Similar to as described above with respect to the knife 220 (of FIGS. 2A-2B), the stability wings 326 may be formed from a shape memory metal wire (which may be coated with PTFE) which extends outward with respect to the elongated body 322 when activated. In other embodiments, the stability wings 326 may be moved into the expanded state position by feeding the wires forming the stability wings 326 in a distal direction from a proximal location, thereby causing these wires to controllably bow out. The degree of expansion of the stability wings 326 may be controlled in some embodiments such that a user can select the degree of expansion, thereby providing variable levels of support. This can be advantageous where, for example, a sufficient level of stability is needed, but cutting device 314 is deployed near fragile and/or vital regions of a body such that too much expansion could come with high risk of harm to the patient. Further, the embodiment with retractable stability wings 326 may be advantageous where it is desirable to save space in a delivery state without sacrificing stability.

Additionally or alternatively, stability can be achieved in other ways, and potentially without the use of stability wings 326. For example, in some embodiments (referring to FIG. 1A), a flat stabilizing platform may be included at the distal end of the cutting device 114, such a flat platform 119 provided on distal portion 118 in the embodiment of FIGS. 1A-1B. In the embodiment best shown by FIGS. 2C-2D, the distal portion 218 of the cutting device 214 is shaped with a widened portion such that it will naturally orient the tissue-engaging surface 216 when in-between two tissue layers. In other embodiments, the elongated body 322 (referring to FIG. 3A) may be made of a braided wire or another material with high rotational stiffness. The elongated body 322 could also be a cannula made of a material with a high degree of rigidity, such as a metal, fiberglass, rigid plastic, etc. The elongated body 322 could additionally and/or alternatively comprise a tab configured to communicate with a groove in the working channel of a delivery device, and/or the delivery device and tubular body 322 may be shaped such that tubular body cannot rotate with respect to the working channel (e.g., both could have an oval shape). Many other methods and devices may be used to provide sufficient stability.

The cutting device 314 may further comprise a tip 330. The tip 330 may be electrified such that the tip 330 creates a cauterizing surface to facilitate the distal motion of the cutting device 314 when moving through tissue. In other embodiments, the tip 330 may comprise a sharp surface or a smooth potentially lubricated surface to achieve this facilitating effect. The tip 330 may further comprise a lumen 332 configured to spray or inject an injectable solution (e.g., a fluid, such as saline) to assist the creation of a submucosal tunnel and/or to facilitate the distal movement of the cutting device 314 when moving through tissue. This lumen 332 may work in conjunction with a cauterizing tip 330 (e.g., the cauterizing tip 330 cuts into the fluid-expanded space) or in isolation to achieve the facilitating effect. In other embodiments, the lumen 332 may be located somewhere proximal of its depicted location on the elongated body 322 to create separation of tissue around the elongated body 322. Multiple lumens 332 or cauterizing surfaces may be included. These embodiments are advantageous, as they may eliminate the requirement of providing separate cutting and/or injecting tools along with cutting device 314.

One procedure for injecting a solution described in U.S. patent application 2011/0208158 to Sigmon et al., which is herein incorporated by reference in its entirety. The injectable solution or gel is preferably a pharmaceutically acceptable solution for use in humans and animals that has minimal tissue reactivity. In some embodiments, the injectable solution has a viscosity greater than about 10,000 cP, and in some embodiments, a viscosity greater than about 30,000 cP and greater than about 50,000 cP. The preferred viscosity for the injectable solution is between about 10,000 to 150,000 cP, and in some embodiment the preferred viscosity for the injectable solution is between about 30,000 cP and about 120,0000 cP, although other viscosities may be used. The viscosity of the injectable solution preferably should be high enough to separate the tissue layers. Non-limiting examples of suitable materials for inclusion in the injectable solution include methylcelluloses, such as carboxymethyl cellulose (CMC) and hydroxypropyl methylcellulose (HPMC), extracellular matrix proteins, elastin, collagen, gelatin, fibrin, agarose, and alginate or mixtures thereof. The injectable solution with be described with reference to CMC although one skilled in the art will understand that other suitable materials may also be used to form the injectable solution.

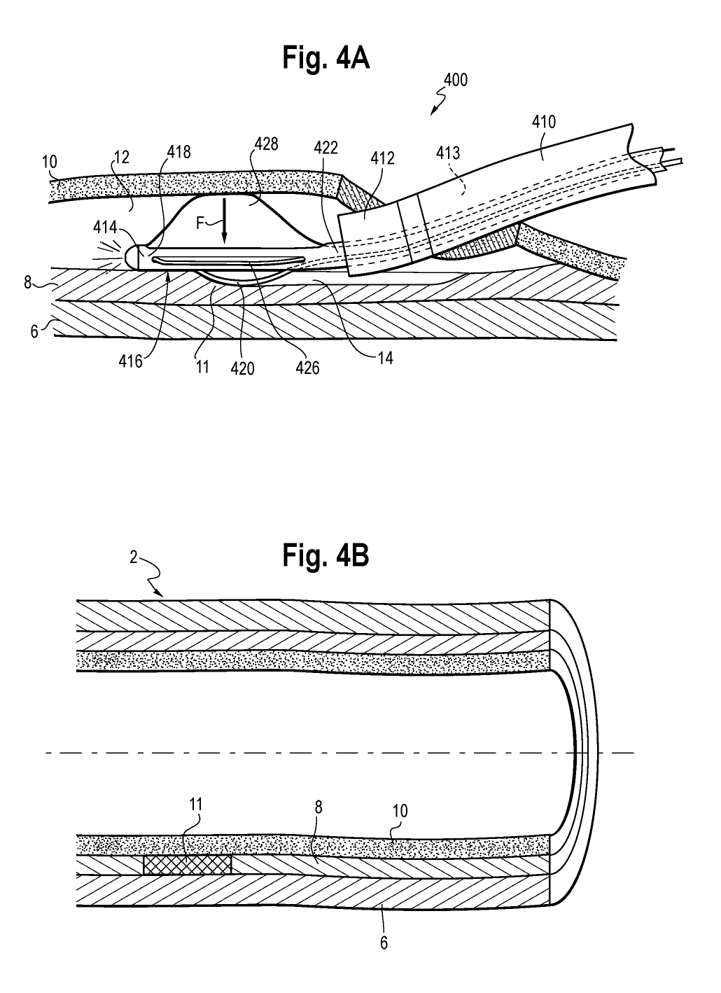

Referring to FIG. 4A, an embodiment of a medical device 400 having a cutting device 414 is shown. The medical device 400 may comprise an expander 428 configured to provide a force (depicted as force F in FIG. 4A) on the cutting device 414. In the depicted embodiment, the medical device 400 comprises a delivery device 410 with a working channel 413 and a cap 412 configured to interact with the cutting device 414. The cutting device 414 comprises a distal portion 418 with a knife 420 and stability wings 426, similar to the knife and stability wings in embodiments described above. The knife 420 is in an active state and is shown as having provided a cut 14 in the top muscle layer 8, wherein the bottom depth of the cut lies above the bottom muscle layer 6 in FIG. 4A. An elongated body 422 of the cutting device 414 is attached to the expander 428, which is configured to expand or grow such that the expander 428 and/or elongated body 422 applies a force on a tissue layer (such as mucosal and submucosal layers 10), as shown. The expander 428 may be, for example, an expandable balloon device, a folded semi-compliant balloon, a shape memory shape, a controllable bowing wire, or any other suitable device. As a result of the expansion of the expander 428, a reaction force, shown in FIG. 4A as force F, may be provided on the cutting device 414 such that the tissue-engaging surface 416 and/or the knife 420 are pressed against the top muscle layer 8. This force F may, for example, ensure that the knife 420 and/or the tissue-engaging surface 416 remain sufficiently engaged with the target area 11 (shown prior to the medical procedure in FIG. 4B) to effectively provide a desired cut, such as the depicted cut 14. This may be advantageous where the operating end of the delivery device 410 and/or the distal portion 418 of the elongated body 422 are a substantial distance from the operator such that the operator cannot provide this desired downward force on the cutting device 414, potentially preventing a suitable cut or making it unduly difficult.

The above figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

We claim:

1. An apparatus for at least partially cutting a tissue layer, the apparatus comprising:
an elongated body with a distal portion;
a tissue-engaging surface at the distal portion; and
a knife proximate to the tissue-engaging surface and having an active state, wherein in the active state, the knife has a cutting portion extending a first distance away from the tissue-engaging surface, wherein the first distance corresponds to a depth of a desired cut within the tissue layer.

2. The apparatus of claim 1, wherein the knife comprises a deflectable portion located proximally of its distal end, and wherein the deflectable portion comprises at least a portion of the cutting portion.

3. The apparatus of claim 1, wherein the tissue-engaging surface faces substantially perpendicular to a longitudinal direction of the distal portion of the elongated body.

4. The apparatus of claim 1, wherein the knife is adjustable to a second state, and wherein in the second state, the cutting portion extends a second distance from the tissue-engaging surface corresponding to a second depth of a desired cut within the tissue layer.

5. The apparatus of claim 1, wherein the knife comprises an inactive state wherein the knife does not extend from the tissue-engaging surface.

6. The apparatus of claim 1, further comprising a stability device extendable away from the body and configured to orient the tissue-engaging surface towards the tissue layer.

7. The apparatus of claim 6, wherein the stability device comprises at least one wing configured to extend away from the elongated body.

8. The apparatus of claim 1, further comprising at least one expander located at the distal portion opposite of the tissue-engaging surface, the expander configured to provide a force for pushing the tissue-engaging surface towards a target tissue.

9. The apparatus of claim 1, further comprising a lumen extending to the distal portion, the lumen being configured to inject a fluid.

10. The apparatus of claim 1, further comprising a cauterizing tip on the distal portion.

11. An cutting device, the cutting device comprising:
an elongated body with a distal end having a tissue-engaging surface; and
a knife having an first state and a second state,
wherein in the first state, the knife extends a first distance away from the tissue-engaging surface, the first distance corresponding with a first desired cutting depth, and
wherein in the second state, the knife extends a second distance away from the tissue-engaging surface, the second distance being less than the first distance.

12. The cutting device of claim 11, wherein the knife comprises a deflectable portion located proximally of its distal end, and wherein the deflectable portion deflects a first amount corresponding with the first distance in the first state and deflects a second amount corresponding with the second distance in the second state.

13. The cutting device of claim 11, the cutting device comprising a stabilizing device configured to orient the tissue-engaging surface towards a tissue layer.

14. The cutting device of claim 13, wherein the stabilizing device comprises at least one wing extending away from the elongated body.

15. The cutting device of claim 11, wherein the tissue-engaging surface faces substantially perpendicular to a longitudinal direction of a distal portion of the elongated body.

16. The cutting device of claim 11, wherein the knife is a cauterizing device.

17. The cutting device of claim 11, wherein the cutting device comprises a lumen extending to the distal end and configured to inject a fluid.

18. The cutting device of claim 11, the cutting device comprising at least one expander configured to provide a force for pushing the tissue-engaging surface towards a target tissue.

19. A method for treating a target area in tissue, the method comprising:
deploying a cutting device with a tissue-engaging surface to the target tissue area such that the tissue-engaging surface contacts the tissue;
deploying a stability device from an elongated body of the cutting device, the stability device being configured to orient the tissue-engaging surface towards the target area in the tissue;
deflecting a cutting portion of a knife, the cutting portion extending a first distance from the tissue-engaging surface, wherein the first distance corresponds with a depth of a desired cut into a target area of tissue; and
moving the cutting device distally and such that the tissue-engaging surface remains in contact with the tissue to form a cut within the tissue with the cutting portion of the knife.

20. The method of claim 19, wherein the method further comprises expanding an expander to provide a force on the tissue-engaging surface towards the target tissue.
